(19) United States
(12) Patent Application Publication
(10) Pub. No.: US 2003/0228263 A1
(43) Pub. Date: Dec. 11, 2003

(54) Propagermanium for treating myeloma bone disease and other bone disorders

(75) Inventor: Anthony Allison, Belmont, CA (US)

Correspondence Address: SWANSON & BRATSCHUN L.L.C., 1745 SHEA CENTER DRIVE, SUITE 330, HIGHLANDS RANCH, CO, 80129, US

(73) Assignee: SurroMed, Inc., Mountain View, CA

(21) Appl. No.: 10/410,866

(22) Filed: Apr. 10, 2003

Related U.S. Application Data
(60) Provisional application No. 60/371,660, filed on Apr. 10, 2002.

(51) Int. Cl. A61K 7/16; A61K 31/28

(52) U.S. Cl. 424/49; 514/492

(57) ABSTRACT

Methods and pharmaceutical compositions containing the organic germanium polymer propagermanium are used for treating bone disorders such as myeloma bone disease, multiple myeloma, Paget's disease, secondary bone cancers, and periodontitis.

### CROSS-REFERENCE TO RELATED APPLICATIONS

This application claims the benefit of U.S. Provisional Application No. 60/371,660, “Propagermanium for Treating Myeloma Bone Disease,” filed Apr. 10, 2002, which is incorporated herein by reference.

### FIELD OF THE INVENTION

The present invention relates generally to compositions and methods for treating myeloma bone disease and related disorders. More particularly, it relates to a pharmaceutical composition containing the organic germanium-containing polymer propagermanium.

### BACKGROUND OF THE INVENTION

Myeloma bone disease is a cancer of antibody-producing plasma cells in the bone marrow. Proliferation of the cancerous plasma cells, referred to as myeloma cells, causes a variety of effects, including lytic lesions (holes) in the bone, decreased red blood cell number, production of abnormal proteins (with attendant damage to the kidney, nerves, and other organs), reduced immune system function, and elevated blood calcium levels (hypercalcemia). When myeloma cells are present at distinct skeletal locations, the disease is referred to as multiple myeloma.

Although responsible for only 1% of all cancers in the United States, with 14,600 new cases reported in 2002, myeloma is the second most common blood cancer and may be increasing in prevalence, particularly among individuals under age 55 (International Myeloma Foundation). Many different treatment options are available or in development, but there is neither a cure nor agreement on an optimal myeloma management regimen. Patients are treated with chemotherapy as well as symptom-specific treatments for one or more of hypercalcemia, increased infection risk, kidney failure, anemia, hyperviscosity of blood, elevated stroke risk, bone destruction and pain, and muscle weakness. Unfortunately, dramatic reduction in the number of myeloma cells does not necessarily translate into longer remissions or survival times, and therapies that were effective before a remission may not prove effective upon relapse of the disease.

One of the most prevalent and significant characteristics of myeloma is the activation of osteoclasts, multinucleated cells that absorb bone, leading to bone thinning, lytic bone lesions, and bone fracture. Lytic bone lesions occur in 70-80% of multiple myeloma patients and are frequently associated with severe bone pain and pathologic fractures. In normal bone functioning, a balance exists between osteoclasts, which resorb bone, and osteoblasts, cells that produce bone. This balance is upset in myeloma patients, and more bone is resorbed than produced. The increased osteoclastic bone resorption occurs adjacent to the myeloma cells and not in areas of normal bone marrow, indicating that the osteoclast activation occurs by a local mechanism. Although it is well accepted that myeloma cells activate osteoclasts, the precise mechanism by which this occurs is unknown. Myeloma cells, in culture, produce or induce production of several osteoclast-activating factors (OAFs) whose specific roles in vivo are yet to be determined. Recently, the chemokine macrophage inflammatory protein-1α (MIP-1α) has been implicated in osteoclast activation in vitro (S. J. Choi et al., “Macrophage inflammatory protein 1-alpha (MIP-1α) is a potential osteoclast stimulatory factor in multiple myeloma,” Blood 96: 671-675, 2000). Therapies addressing mechanisms involving OAFs are presently under development.

Currently, bone indications of multiple myeloma are treated primarily with bisphosphonates, a class of chemicals that inhibits osteoclast activity or osteoclast attachment to bone surface and eventually leads to osteoclast cell death. They may also affect myeloma cells directly. Bisphosphonates are administered by infusion. Third-generation bisphosphonates are currently under development, but even improved versions of the drugs may-have potential side effects including hypocalcemia, kidney damage, and increased pain. Bisphosphonates do not completely block the bone destruction process, and patients eventually develop new bone lesions. An alternative therapy for bone destruction in multiple myeloma that can be administered orally would be highly beneficial.

### BRIEF DESCRIPTION OF THE FIGURES

FIG. 1 is a schematic diagram of the chemical structure of a monomeric unit of the polymer propagermanium.

FIG. 2 shows the effect of propagermanium on formation of osteoclasts, as measured by TRAP activity, in mouse bone marrow cells stimulated with MIP-1α.

FIG. 3 shows the effect of propagermanium on formation of osteoclasts in human bone marrow cells stimulated with MIP-1α.

### DETAILED DESCRIPTION OF EMBODIMENTS OF THE INVENTION

Various embodiments of the present invention provide methods and pharmaceutical compositions for treating myeloma bone disease, multiple myeloma, and related bone disorders. In general, any bone disorder in which osteoclasts are activated by chemokines such that a normal balance between bone resorption by osteoclasts and bone formation by osteoblasts is disrupted, or in which reduction of osteoclast activity or inhibition of bone resorption would be beneficial, can be treated by methods of the invention.

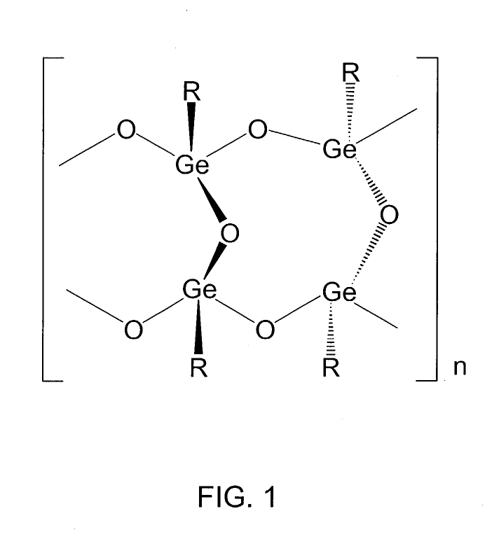

In one embodiment of the invention, a pharmaceutical composition containing a therapeutically effective amount of the organic germanium compound propagermanium is administered to a subject suffering from myeloma bone disease or other bone disorder. Propagermanium, or 3-oxygermylpropionic acid polymer, has a chemical formula [(O_{1/2})₃GeCH₂CH₂CO₂H]ₙ, where n is an integer greater than 1, and a structure as shown in FIG. 1. Suitable weight-average molecular weights are between 10⁴ and 10⁵, e.g., 9.29±5.72×10⁴, corresponding to weight-average degrees of polymerization n of approximately 548±337. Propagermanium can be obtained from Sanwa Kagaku Kenkyusho, Ltd (Nagoya, Japan), a company that manufactures the drug as a treatment for hepatitis B and sells it as a 10 mg capsule under the name SEROCION®.

As used herein, a “therapeutically effective” amount refers to an amount that produces a reduction in symptom severity (e.g., bone destruction), delay in disease development, decrease in bone resorption by osteoclasts, decrease in number of osteoclasts produced from osteoclast precursors or attracted to bone surfaces, or any other measurable effect on the progression or development of the bone disorder. Effective amounts can be determined using routine optimization techniques and depend on the particular disorder treated, the condition of the patient, the route of drug administration, the particular formulation, and other factors known to those of skill in the art. General guidance for determining a therapeutically effective amount can often be gained from experiments performed in relevant animal models. Note that treatment of the bone disorder with embodiments of the pharmaceutical composition may include treatment of both the symptoms of the disorder and its underlying mechanism. For example, propagermanium may affect myeloma cells directly or indirectly as well as the resulting bone destruction.

The propagermanium-containing composition can be administered orally as, e.g., a tablet, capsule, grain, or powder, any of which may contain a suitable high- or low-molecular-weight carrier in addition to the propagermanium. High-molecular-weight carriers such as proteins or polymers may help to stabilize the propagermanium in the composition. Examples of suitable carriers include, without limitation, sugars such as lactose, sucrose, dextran, sorbitol, fructose, and glucose; modified celluloses such as hydroxypropylcellulose; naturally occurring polymers such as serum, serum albumin, and pepsin; and manufactured polymers such as polyvinylpyrrolidone. The pharmaceutical composition can also include an inert filling, binder, or disintegrator. Typical concentrations of carrier are between about 0.001 and about 1000 parts per part propagermanium by weight or between about 0.01 and about 200 parts by weight. Alternatively, the pharmaceutical composition can contain between about 0.01 weight percent and about 1 weight percent of propagermanium and between about 0.5 weight percent and about 10 weight percent of the high-molecular-weight carrier, with the remaining composition provided by low-molecular-weight substances.

Additional propagermanium compositions suitable for use in embodiments of the present invention are provided in the following US patents, all issued to Sawai et al., and all incorporated herein by reference: U.S. Pat. No. 4,889,715; U.S. Pat. No. 5,240,700; U.S. Pat. No. 5,260,056; U.S. Pat. No. 5,336,688; U.S. Pat. No. 5,340,806; and U.S. Pat. No. 5,532,272.

In one embodiment of the invention, the pharmaceutical composition is administered orally to a patient with a bone disorder at a dose of between about 1 mg/day and about 1500 mg/day of propagermanium. In another embodiment, the pharmaceutical composition is administered orally at a dose of between about 10 mg/day and about 150 mg/day of propagermanium. Administration can occur once per day or in divided doses, e.g., three times per day, once after each meal. The complete daily dose can be divided into any number of individual doses per day, based in part on the known pharmacokinetics of the drug. In general, an appropriate dose may vary based upon the extent and severity of disease progression, the subject's age, weight, sex, and general physical condition, and other factors known to those of skill in the art.

In an additional embodiment, the composition is delivered orally at a dose of between about 30 mg/day and about 100 mg/day of propagermanium. This dose exceeds the current value (30 mg/day) administered for the treatment of hepatitis. For example, propagermanium can be administered in a dose of about 50 mg twice per day or about 30 mg three times per day. For treating hepatitis, the daily dose of propagermanium is limited to protect against potential hepatotoxic effects of the drug. Typically, liver damage can be detected by elevated levels of transaminase (liver enzyme) in the blood. Because hepatitis patients already have elevated transaminase levels, it is difficult to detect liver damage caused directly by propagermanium. A low dose of propagermanium is, therefore, used as a precaution. Myeloma patients typically do not have elevated transaminase levels, and there is no risk that hepatotoxic effects will go undetected. For this reason, higher doses of propagermanium are possible and, in some cases, desirable for treating myeloma and other bone disorders than for treating hepatitis.

In an alternative embodiment, the pharmaceutical composition is administered in combination with other drugs such as anti-cancer drugs or drugs that treat symptoms of myeloma bone disease. Other administration methods, such as intravenous or parenteral, by a series of injections or continuous infusion over an extended period of time, or external, may also be used. Depending on the formulation, a pharmaceutically acceptable vehicle may be included in the pharmaceutical composition. Doses for alternative delivery routes are similar to those provided above for oral administration.

One embodiment of the present invention is a pharmaceutical composition containing a therapeutically effective amount of propagermanium. The amount is effective for treating bone disorders such as myeloma bone disease, Paget's disease, and secondary bone cancers (cancers, e.g., prostate or breast, that that have metastasized to bone), and may exceed the dose of propagermanium typically administered to hepatitis patients (30 mg/day). For example, the amount may be greater than 10 mg, greater than 20 mg, greater than 30 mg, or higher, so that, depending on how many times the composition is administered, a patient receives a total daily dose that is greater than 30 mg. The composition may also contain low-molecular-weight and high-molecular-weight carriers.

Although the present invention is not limited to any particular mechanism or theory, propagermanium may ameliorate bone loss in myeloma bone disease by preventing the chemotactic and other responses of osteoclasts to the CC chemokine macrophage inflammatory protein-1α (MIP-1α), which is produced by myeloma cells in vivo. Chemokines are chemotactic cytokines that are released by a wide variety of cells to attract leukocytes. There are four subfamilies of chemokines, classified on the basis of their N-terminal amino acid sequence and the type of leukocyte they attract. CC or α chemokines, which are potent chemoattractants of monocytes, have adjacent first and second N-terminal cysteine residues, unlike the other three chemokine subfamilies, which have either one N-terminal cysteine residue or multiple cysteine residues separated by other amino acids.

CC chemokines include macrophage inflammatory proteins 1α and 1β (MIP-1α, MIP-1β), RANTES (regulated on activation, normal T-cell expressed and secreted protein), MCP-1, MCP-2, and MCP-3, among others. The mechanism of CC chemokine action involves initial binding to specific seven-transmembrane-domain G-protein-coupled receptors on target cells and activation of a signal transduction system involving activation of the small GTPase Rho. On binding their cognate ligands, chemokine receptors transduce an intracellular signal through the associated G protein. Although the receptors of different chemokines differ, there is overlap in the signal transduction mechanisms of monocyte-macrophage cells for all CC chemokines. CCR1 and CCR5 are the primary receptors for MIP-1α.

MIP-1α has been shown to induce formation of osteoclasts from precursor cells in human marrow cultures (J. H. Han et al., “Macrophage inflammatory protein 1-alpha is an osteoclastogenic factor in myeloma that is independent of RANK ligand,” Blood 97: 3349-3353, 2001) and to be chemotactic for osteoclasts (K. Fuller et al., “Macrophage inflammatory protein 1-α and IL-18 stimulate the motility but suppress the resorption of isolated rat osteoclasts,” J. Immunol. 154: 6065-6072, 1995). It is increased in marrow plasma from myeloma patients with active disease and reduced to normal levels in patients in remission (S. J. Choi et at., “Macrophage inflammatory protein 1-alpha (MIP-1α) is a potential osteoclast stimulatory factor in multiple myeloma,” Blood 96: 671-675, 2000). In a mouse model of human myeloma bone disease, antisense inhibition of MIP-1α was shown to decrease both bone destruction and tumor burden (S. J. Choi et al., “Antisense inhibition of macrophage inflammatory protein 1-α blocks bone destruction in a model of myeloma bone disease,” J. Clin. Invest. 108: 1833-1841, 2001).

In embodiments of the present invention, propagermanium may inhibit the effect of MIP-1α on osteoclasts, thereby decreasing differentiation and chemotaxis of osteoclasts and the resulting bone resorption. It may also inhibit the effect of other CC chemokines on osteoclasts. Propagermanium has been shown to inhibit the chemotactic response of monocytes to the CC chemokine MCP-1, most likely via glycosylphosphatidylinositol (GPI)-anchored proteins (S. Yokochi et al., “An anti-inflammatory drug, propagermanium, may target GPI-anchored proteins associated with an MCP-1 receptor, CCR2,” J. Interferon Cytokine Res., 21: 389-398, 2001). The receptor for MCP-1 on monocytes, CCR2, is coupled to GPI-anchored proteins, which are restricted to the outer leaflet of the cell membrane and integrated to the membrane via phosphatidylinositol (PI). In this study, propagermanium did not appear to interact directly with the chemokine receptor, but rather associated with GPI-anchored proteins coupled to the chemokine receptor, inhibiting the signal activation system required for chemotaxis of the cell. It was proposed that binding of propagermanium to a GPI-anchored protein colocalized with CCR2 induces a signal that regulates CCR2-mediated intracellular signaling pathways via the small GTPase Rho, thereby inhibiting chemotaxis.

In embodiments of the present invention, propagermanium may, by inhibiting the chemokine receptor transduction mechanism, inhibit osteoclast chemotaxis by a similar mechanism by which it inhibits chemotaxis of monocytes in response to MCP-1. Propagermanium has been shown to suppress the recruitment of macrophages into the liver (S. Yokochi et al., “Hepatoprotective effect of propagermanium on Corynebacterium parvum and lipopolysaccharide-induced liver injury in mice,” Scand. J. Immunol. 48: 183-191, 1998; and Y. Ishiwata et al., “Protection against Concanavalin A-induced murine liver injury by the organic germanium compound, propagermanium,” Scand. J. Immunol. 48: 605-614, 1998), indicating that it has activity against several chemokines involved in chemotaxis.

An effect of propagermanium on human and mouse osteoclasts in vitro is shown and described in the examples below. These results indicate that propagermanium specifically inhibits formation of osteoclasts from osteoclast precursor cells when stimulated by MIP-1α. Propagermanium is thought to inhibit both osteoclast differentiation from osteoclast precursors in response to MIP-1α and chemotaxis of osteoclasts in response to MIP-1α. These in vitro results indicate that propagermanium may decrease osteoclastic bone destruction in vivo.

Although the present invention is not limited to any particular mechanism, the effect of germanium-containing compounds on signal transduction pathways may be analogous to the well-known effects of silicon on cell membrane-associated proteins. Similarly to silicon oxide, germanium oxide groups may form hydrogen bonds with phospholipids in the cell membrane, affecting the function of membrane-associated proteins such as receptor tyrosine kinase. Propagermanium may enter macrophages through endocytic vacuoles, which may be observed, e.g., using x-ray spectrometry.

Additional information on dosage and in vivo effects of propagermanium on myeloma bone disease can be obtained by administering propagermanium to animal models of human multiple myeloma, such as transgenic mice or SCID (severe combined immunodeficient) mice. For examples and discussions of existing murine models of multiple myeloma, see K. Gado et al., “Mouse plasmacytoma: an experimental model of human multiple myeloma,” Haematologica 86: 227-236, 2001, which is incorporated herein by reference. All of the references cited in the Gado et al. reference are also incorporated herein by reference. Any additional suitable animal models may be employed in determining appropriate doses in embodiments of the present invention.

Although embodiments of the present invention have been described primarily with respect to treating myeloma bone disease and multiple myeloma, it will be apparent to one of skill in the art that propagermanium may be used to treat any bone disorder in which bone resorption by osteoclasts exceeds bone production by osteoblasts, or in which it would be beneficial to reduce bone resorption by osteoclasts or chemotaxis of osteoclasts. One example is Paget's disease, in which both osteoclasts and osteoblasts exhibit increased activity, yielding bone that is structurally unsound. Symptoms of Paget's disease include bone pain, bone deformity, and skeletal fragility. The fundamental and initial abnormality in Paget's disease resides in abnormally large osteoclasts and their excessive bone resorption, which triggers increased bone formation by normal osteblasts. Inhibiting osteoclast activity by administering propagermanium may also, therefore, decrease osteoblast activity.

Other examples of bone disorders that are treated in methods of the present invention include secondary bone cancers, which originate in other parts of the body before spreading to the bone. While virtually all cancers can spread to bone, bone metastases are particularly common in breast, lung, prostate, kidney, and thyroid cancers. Secondary bone cancers are currently treated with bisphosphonates.

In an additional embodiment of the invention, a pharmaceutical composition containing propagermanium is administered to treat periodontal disease (periodontitis), a bacterial-associated inflammatory disease of the supporting tissues of the teeth. In severe cases of the disease, bone erosion by osteoclasts occurs. In this embodiment, the propagermanium-containing composition is administered to reduce or delay bone loss. The dental composition can be administered topically, e.g., as an oral rinse, oral cream, mouthwash, toothpaste, lozenge, chewing gum, gel, or any other orally absorbable dental formulation. Depending upon the particular formulation, the composition is kept in the mouth for different periods of time and in different manners (e.g., held against the teeth under pressure, swished in the mouth, brushed against teeth, chewed or sucked, etc.). Lower-molecular-weight formulations of propagermanium may be desirable to increase diffusion of propagermanium into the tissue. Suitable doses of propagermanium vary based on its molecular weight and the mode of application, in addition to factors discussed above. Typical doses for treating periodontitis are between about 2 mg/day and about 20 mg/day. When administered in divided doses (e.g., morning and evening), each unit (lozenge, fixed amount of dental rinse, squirt of paste, etc.) of the dental composition contains at least about 1 mg propagermanium. If administered once per day, the dental composition contains between about 2 mg and about 20 mg of propagermanium.

When administered to treat periodontitis, propagermanium may be added to oral rinses or toothpastes containing alcohol, fluoride, or other active ingredients. Additional flavors, emollients, and carriers may also be included.

### WORKING EXAMPLES

The following examples illustrate embodiments of the invention without limiting the embodiments to the details disclosed.

### Working Example 1

### Effect of Propagermanium on Formation of Mouse Osteoclasts

Murine osteoclast precursor cells were treated with MIP-1α with and without propagermanium and the osteoclast formation quantified. Results show an inhibition of osteoclast formation by propagermanium.

Bone marrow cells (10⁶ cells/culture) from C57B1 mice were isolated and cultured for osteoclast-like multinucleated cell formation in the presence or absence of 1, 10, or 100 ng/ml MIP-1α, 10⁻¹¹ M 1,25-(OH)₂D₃ (vitamin D), and varying concentrations of anti-human MIP-1α antibody (20 or 100 ng/ml), anti-CCR1 receptor antibody (20 or 100 ng/ml), anti-CCR5 receptor antibody (20 or 100 ng/ml), and 1 μg/ml propagermanium. Twelve different culture compositions were each prepared in four wells of a 48-well plate. After approximately six or seven days, the cultures were stained for the enzyme tartrate-resistant acid phosphatase (TRAP), an enzyme secreted into the circulation by osteoclasts during bone resorption, using an acid phosphatase staining kit (Sigma). TRAP-positive multinucleated cells containing three or more nuclei (assumed to be osteoclasts) were counted with an inverted microscope. The readout can be either the total amount of TRAP (TRAP activity) or the number of osteoclasts.

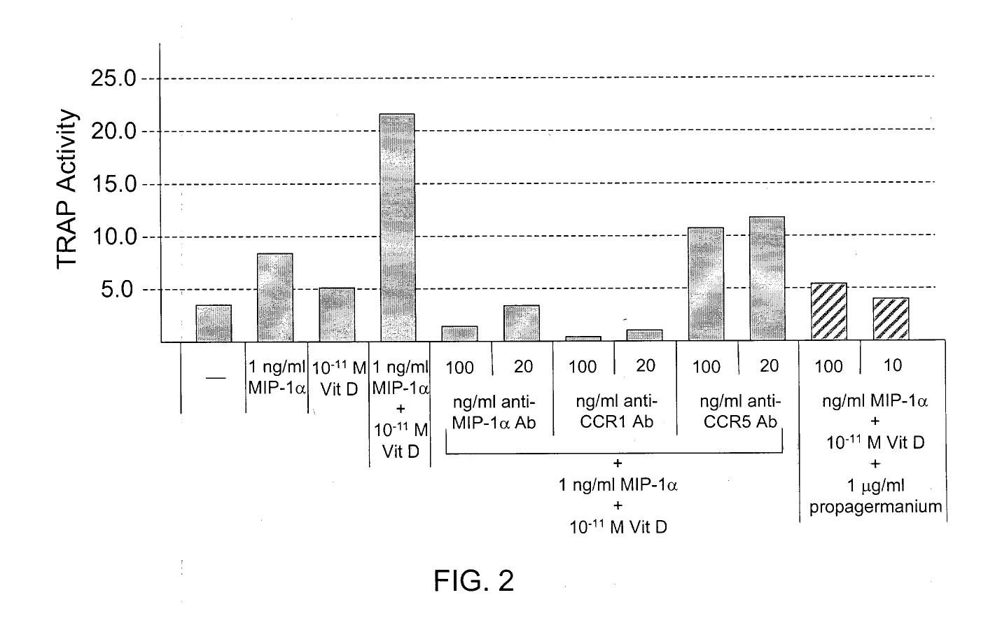

Results are presented in FIG. 2, which plots TRAP activity in each well of the specified type. Substantial increases in osteoclast formation were observed with MIP-1α and vitamin D in combination. Dose-dependent decreases in osteoclast formation were seen with additions of the antibodies to MIP-1α and the MIP-1α receptors CCR1 and CCR5. Propagermanium reduced the number of osteoclasts formed in the presence of two different concentrations of MIP-1α by greater than a factor of four. These results indicate that propagermanium inhibits the formation of mouse osteoclasts stimulated by MIP-1α.

### Working Example 2

### Effect of Propagermanium on Human Osteoclasts

Human osteoclast precursor cells were treated with MIP-1α with and without propagermanium and the osteoclast formation quantified. Results show a dose-dependent inhibition of osteoclast formation by propagermanium.

Human long-term marrow cultures were performed as described in D. E. Hughes et al., “Estrogen promotes apoptosis of murine osteoclasts mediated by TGF-β,” Nature Medicine 2: 1132-1136, 1996, which is incorporated herein by reference. Briefly, human bone marrow nonadherent mononuclear cells from normal volunteers were cultured at 10⁶ cells/ml in α-minimum essential medium and 20% horse serum with or without 10⁻⁸ M 1,25-(OH)₂D₃ (vitamin D), 200 pg/ml MIP-1α, 20 ng/ml anti-human MIP-1α antibody, and varying concentrations of propagermanium. Five wells of each of ten different culture conditions were prepared in a 96-well plate. Each well contained 10⁵ cells and the desired combination of reagents. Half of the culture medium was changed weekly. After approximately three weeks, the cultures were harvested and stained with the 23c6 monoclonal antibody, which identifies the osteoclast vitronectin receptor, and the 23c6-positive multinucleated cells counted. The 23c6 assay is more specific than the TRAP assay (used in Example 1 for mouse osteoclasts) for human osteoclasts.

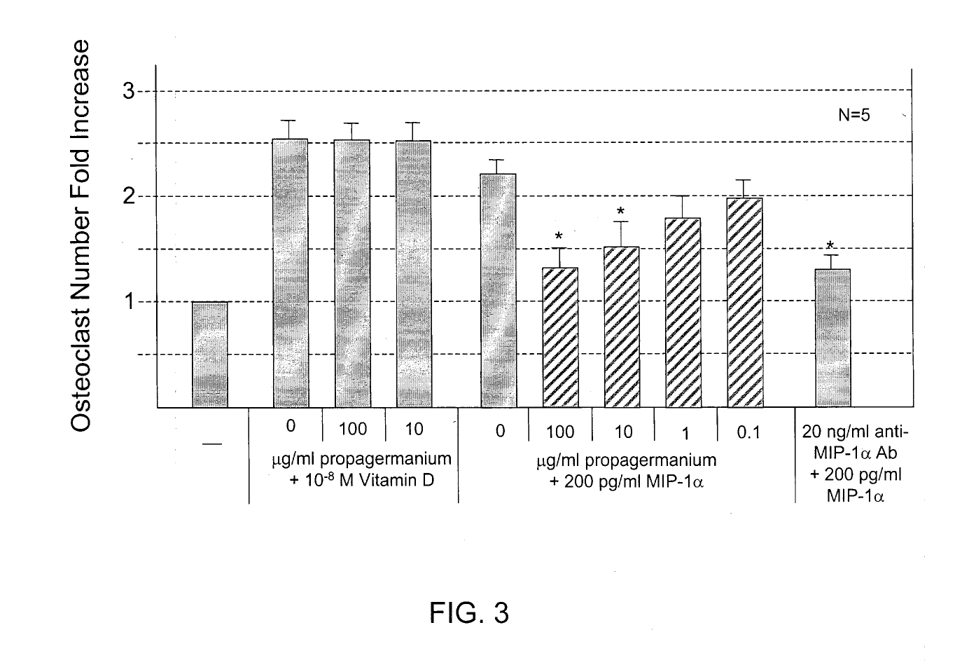

Results are shown in FIG. 3, a plot of fold increase in osteoclasts over the number of osteoclasts formed in untreated marrow for each set of wells. Well conditions are indicated below the bars. Results are reported as the mean±S.E. for five replicate samples and were compared by Student's t test. Results were considered significantly different for p<0.05. As shown, both MIP-1α and vitamin D alone caused a greater than two-fold increase in the number of osteoclasts formed. Propagermanium had no effect on osteoclast formation in response to vitamin D stimulation but caused a dose-dependent reduction in the number of osteoclasts formed in response to MIP-1α stimulation. These results indicate that propagermanium specifically inhibits the effect of MIP-1α on osteoclasts but does not generally affect osteoclast formation. The highest dose of propagermanium inhibited the effect of MIP-1α at a level comparable to that of the anti-human MIP-1α antibody. The doses of propagermanium inhibiting osteoclast formation are comparable to those found in the circulation of treated patients.

It should be noted that the foregoing description is only illustrative of the invention. Various alternatives and modifications can be devised by those skilled in the art without departing from the invention. Accordingly, the present invention is intended to embrace all such alternatives, modifications and variances which fall within the scope of the disclosed invention.

What is claimed is:

1. A method for treating a subject having a bone disorder, comprising administering to said subject a pharmaceutical composition comprising a therapeutically effective amount of propagermanium.

2. The method of claim 1, wherein said bone disorder is myeloma bone disease.

3. The method of claim 1, wherein said bone disorder is chosen from at least one of Paget's disease, secondary bone cancer, and periodontitis.

4. The method of claim 1, wherein said propagermanium has a weight-average molecular weight of approximately 9.3×10^{4.}

5. The method of claim 1, wherein said pharmaceutical composition is administered orally.

6. The method of claim 1, wherein said pharmaceutical composition is at least one of a tablet, capsule, oral rinse, and toothpaste.

7. The method of claim 1, wherein said pharmaceutical composition further comprises a carrier.

8. The method of claim 1, wherein said pharmaceutical composition is administered at a dose of between about 1 mg/day and about 1500 mg/day of said propagermanium.

9. The method of claim 1, wherein said pharmaceutical composition is administered at a dose of between about 10 mg/day and about 150 mg/day of said propagermanium.

10. The method of claim 1, wherein said pharmaceutical composition is administered at a dose of between about 30 mg/day and about 100 mg/day of said propagermanium.

11. The method of claim 1, wherein said pharmaceutical composition is administered at a dose of between about 2 mg/day and about 20 mg/day of said propagermanium.

12. A method for inhibiting bone resorption in a subject comprising administering to said subject a therapeutically effective amount of propagermanium.

13. A dental composition comprising a therapeutically effective amount of propagermanium.

14. The dental composition of claim 13, wherein said therapeutically effective amount is between about 1 mg and about 20 mg.
